(19) United States
(12) Patent Application Publication
(10) Pub. No.: US 2009/0220046 A1
(43) Pub. Date: Sep. 3, 2009

(54) COLLIMATOR DEVICE FOR RADIOTHERAPY AND RADIOTHERAPY APPARATUS USING THE SAME

(75) Inventors: Young Hoon JI, Seoul (KR); Dong Han LEE, Uijeongbu Si (KR); Mon Sik CHOI, Uijeongbu Si (KR); Seung Woo PARK, Seoul (KR); Yoon Jong KIM, Seoul (KR)

Correspondence Address: CANTOR COLBURN, LLP, 20 Church Street, 22nd Floor, Hartford, CT, 06103, US

(73) Assignee: KOREA INSTITUTE OF RADIOLOGICAL & MEDICAL SCIENCES, Seoul (KR)

(21) Appl. No.: 12/361,153

(22) Filed: Jan. 28, 2009

(30) Foreign Application Priority Data
Feb. 29, 2008 (KR) 10-2008-0019297

(51) Int. Cl. A61N 5/10; G21K 1/04

(52) U.S. Cl. 378/65; 378/150

(57) ABSTRACT

Provided is a collimator device for radiotherapy including: a body including a first through unit and disposed on a path of high energy radiation which in use is irradiated toward a patient's treatment part; a frame including a through hole corresponding to the first through unit and slidably installed in the body; a plurality of multi-leaf collimators (MLCs) slidably installed in the through hole and including radiation shields; a servo motor coupled to the body and the frame in a power manner so as to slidingly move the frame with respect to the body; and a motor controller externally receiving position displacement data regarding a motion of the patient's treatment part due to a patient's breathing and generating a signal for controlling the driving of the servo motor so that the MLCs follow the patient's treatment part and continuously apply radiation to the patient's treatment part based on the position displacement data.

### CROSS-REFERENCE TO RELATED PATENT APPLICATION

This application claims the benefit of Korean Patent Application No. 10-2008-0019297, filed on Feb. 29, 2008, in the Korean Intellectual Property Office, the disclosure of which is incorporated herein in its entirety by reference.

### BACKGROUND OF THE INVENTION

1. Field of the Invention

The present invention relates to a collimator device for radiotherapy and a radiotherapy apparatus, and more particularly, to a collimator device for radiotherapy installed in a radiotherapy apparatus used for treating a cancer patient, by using a patient's treatment part continuously and precisely irradiated while a physiologically moving treatment part is followed, and the radiotherapy apparatus including the collimator device.

2. Description of the Related Art

In modern times, people have difficulties in maintaining good health due to stress and irregular meals present in our ever complex society. In particular, it is very common for people in modern times to die from a vicious tumor, i.e. cancer. The amount of attacks of cancer is also increasing socially and thus effective counter measures are rapidly required. Therefore, in modern times, methods of treating cancer, and in particular, radiotherapy, have become important points of interest.

Two core elements are necessary for successful radiotherapy on tumor. That is, first, radiation is required to be precisely applied to the tumor, and second, a planned radiation dose should be identical to the radiation dose that is actually applied.

A variety of displacement errors must be reduced in order to precisely apply radiation to the tumor. Displacement errors caused by a patient's body may be classified into three categories: a position related organ motion error, a gap fraction organ motion error, and an internal fraction organ motion error.

The position related organ motion error occurs due to changes in movements of a patient's internal organs according to a patient's posture, such as standing or lying down, while the patient is being treated. The position related organ motion error may be reduced by imaging the patient's posture when treating the patient and planning a treatment position.

The gap fraction organ motion error occurs due to changes in a corresponding organ and its neighboring organs according to how much a bladder, a rectum, or a stomach is filled. The gap fraction organ motion error may be removed by making the patient's condition the same as when the treatment is planned and when the patient is actually treated.

The internal fraction organ motion error occurs due to changes in positions of a corresponding organ and its neighboring organs according to breathing or heartbeat. The internal fraction organ motion error physiologically occurs frequently to a living body. In particular, breathing has a significant effect and thus the internal fraction organ motion error is a serious problem to organs influenced by a diaphragmatic respiration. Thus, the internal fraction organ motion error may be removed by tracing an external anatomic motion according to the patient's breath and irradiating a specific part of an internal organ according to a change in the position of the specific part.

The inventors of the present invention have invented devices detailed in Korean Patent Nos. 0706758 and 0740340.

However, if the above devices are used to irradiate a patient's treatment part, a radiation opening and closing device is opened only when an organ is positioned in a specific part, which increases the time taken to actually treat the patient.

Meanwhile, a patient's treatment part is irradiated by a radiotherapy apparatus to which a shield is attached in order to protect a normal tissue of the patient's treatment part. The shield includes a generally used Lipowitz metal shield or a multi-leaf collimator (MLC). The Lipowitz metal shield needs to be made using an alloy block which requires one or two days for manufacturing, whereas an MLC does not need time for manufacturing an additional shield, and can be more easily manufactured into various irradiation surfaces compared to the alloy block. However, conventional MLCs are expensive and do not work together with various devices required for applying radiation.

### SUMMARY OF THE INVENTION

The present invention provides a collimator device for radiotherapy that continuously and precisely irradiates a patient's treatment part while following the motion of a patient's internal organ, and a radiotherapy apparatus.

According to an aspect of the present invention, there is provided a collimator device for radiotherapy comprising: a body including a first through unit and disposed on a path of high energy radiation which in use is irradiated toward a patient's treatment part; a frame including a through hole corresponding to the first through unit and slidably installed in the body; a plurality of multi-leaf collimators (MLCs) slidably installed in the through hole and including radiation shields; a servo motor coupled to the body and the frame in a power manner so as to slidingly move the frame with respect to the body; and a motor controller externally receiving position displacement data regarding a motion of the patient's treatment part due to the patient's breathing and generating a signal for controlling the driving of the servo motor so that the MLCs follow the patient's treatment part and continuously apply radiation to the patient's treatment part based on the position displacement data.

The body may comprise two guide rails, wherein the frame is slidable along the two guide rails.

The radiation shields may be formed of carbon steel or tungsten alloy.

The MLCs may be manually manipulated.

The MLCs may be slidable by the correlation coupling of adjacent MLCs and each having a non linear cross sectional structure.

The collimator device may further comprise: a template establishing the shape of a radiation pass area of the MLCs and formed of an acrylic material.

The frame may be movably coupled to move in a direction with respect to the body, wherein the servo motor is installed in the frame and is coupled to the frame in a powered manner so as to move the frame in the direction in accordance with the body.

The body may comprise a sliding member movably coupled to the body in a first direction, wherein the frame is movably coupled to the sliding member in a second direction perpendicular to the first direction with respect to the sliding member, wherein the servo motor comprises a first servo motor installed in the body and coupled to the sliding member in a powered manner so as to move the sliding member in the first direction with respect to the body, and a second servo motor installed in the sliding member and coupled to the frame in a powered manner so as to move the frame in the second direction with respect to the sliding member.

According to an aspect of the present invention, there is provided a radiotherapy apparatus comprises: a device for applying radiation; a body including a first through unit, disposed on a path of high energy radiation which in use is irradiated toward a patient's treatment part, and coupled to the device for applying radiation; a frame including a through hole corresponding to the first through unit and slidably installed in the body; a plurality of MLCs slidably installed in the through hole and including radiation shields; a servo motor coupled to the body and the frame in a powered manner so as to slidingly move the frame with respect to the body; and a motor controller externally receiving position displacement data regarding a motion of the patient's treatment part due to a patient's breathing and generating a signal for controlling the driving of the servo motor so that the MLCs follow the patient's treatment part and continuously apply radiation to the patient's treatment part based on the position displacement data.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic block diagram of a radiotherapy apparatus according to an embodiment of the present invention;

FIG. 2 schematically illustrates the elements of the radiotherapy apparatus shown in FIG. 1 according to an embodiment of the present invention;

FIG. 3 is a perspective view of a collimator device for radiotherapy shown in FIG. 2 according to an embodiment of the present invention;

FIG. 4 is a plan view of the collimator device for radiotherapy shown in FIG. 3 according to an embodiment of the present invention;

FIG. 5 is a diagram for explaining the coupling structure of a plurality of collimators of the collimator device for radiotherapy shown in FIG. 3 according to another embodiment of the present invention;

FIGS. 6A through 10C illustrate results of tests conducted to explain an effect of the collimator device for radiotherapy shown in FIG. 4 according to an embodiment of the present invention; and

FIG. 11 is a schematic perspective view of a collimator device for radiotherapy according to another embodiment of the present invention.

### DETAILED DESCRIPTION OF THE INVENTION

Hereinafter, the present invention will be described in detail by explaining embodiments of the invention with reference to the attached drawings.

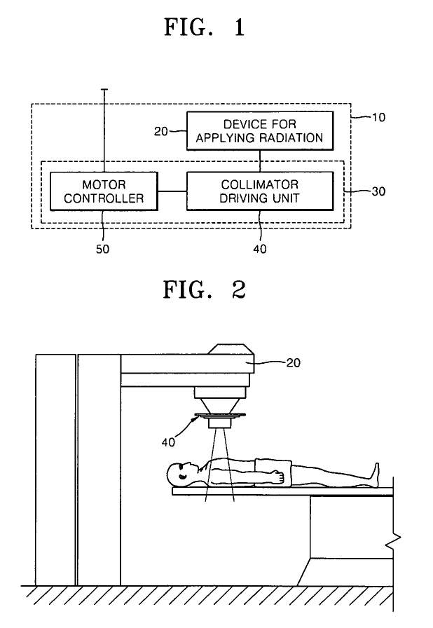

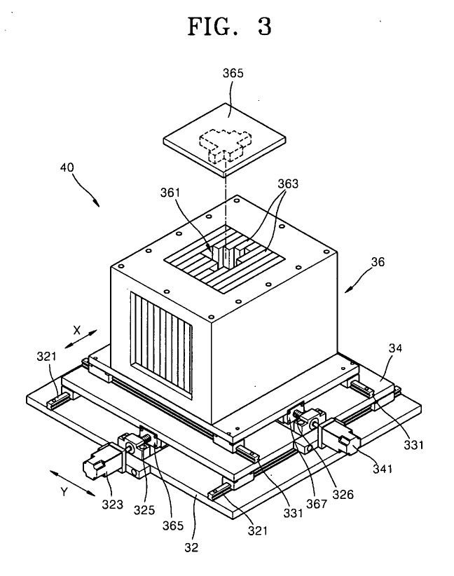

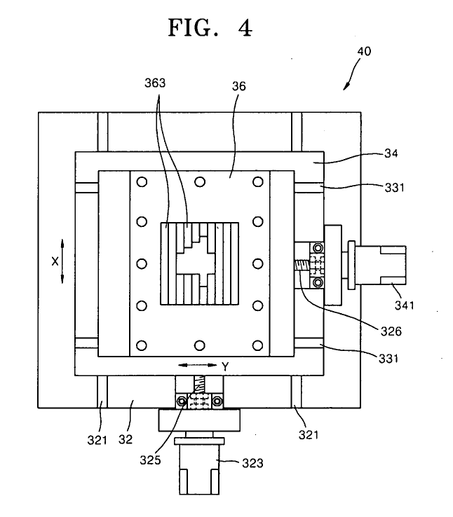

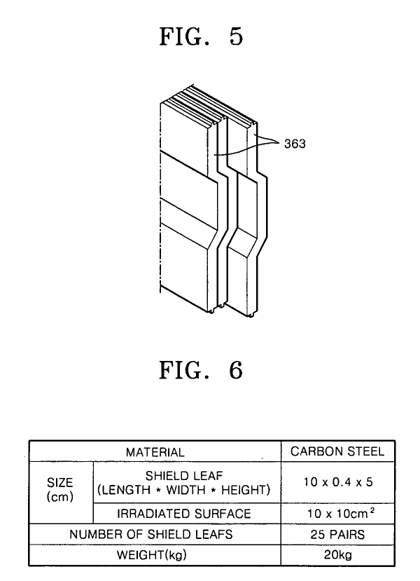

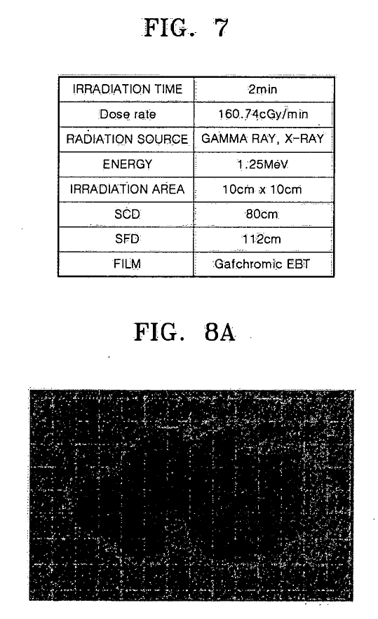

FIG. 1 is a schematic block diagram of a radiotherapy apparatus 10 according to an embodiment of the present invention. FIG. 2 schematically illustrates the elements of the radiotherapy apparatus 10 shown in FIG. 1 according to an embodiment of the present invention. FIG. 3 is a perspective view of a collimator device 30 for radiotherapy shown in FIG. 2 according to an embodiment of the present invention. FIG. 4 is a plan view of the collimator device 30 for radiotherapy shown in FIG. 3 according to an embodiment of the present invention. FIG. 5 is a diagram for explaining the coupling structure of a plurality of collimators of the collimator device 30 for radiotherapy shown in FIG. 3 according to another embodiment of the present invention.

Referring to FIGS. 1 through 5, the radiotherapy apparatus 10 comprises a device for applying radiation 20 and the collimator device 30 for radiotherapy.

The device for applying radiation 20 applies radiation treatment to a patient's part that is to be treated. The device for applying radiation 20 is a device for accelerating electrons or particles and generating and applying radiation, generally used in physics or medical science, and its structure and principle are known and thus detailed description thereof will not be provided here.

The collimator device 30 for radiotherapy comprises a collimator driving unit 40 and a motor controller 50.

The collimator driving unit 40 comprises a body 32, a sliding member 34, a frame 36, a first servo motor 323, and a second servo motor 341.

The body 32 is fixed to the device for applying radiation 20. The body 32 includes a first through unit (not shown). The first through unit is disposed on a path of high energy radiation which in use is irradiated toward the patient's treatment part in the device for applying radiation 20. The body 32 comprises two guide rails 321. The body 32 is formed of a metal material, such as carbon steel or aluminum alloy. However, the present invention is not limited thereto and the body 32 may be formed of any materials for supporting the frame 36. The first servo motor 323 is installed in the body 32.

The sliding member 34 is movably installed in a direction with respect to the body 32. The sliding member 34 is slidably installed with respect to the body 32 in a first direction X along the guide rails 321 disposed on the body 32 as shown in FIG. 3. A second through unit (not shown) corresponding to the first through unit disposed on the body 32 is disposed in the sliding member 34. In the present embodiment, the sliding member 34 is coupled to the first servo motor 323 in a powered manner by using a ball screw 325 and a ball nut 365 that are widely used to convert rotational motion into rectilinear motion. In more detail, the ball screw 325 is fixed to an output axis of the first servo motor 323, and the ball nut 365 into which the ball screw 325 is screwed is coupled to the sliding member 34, so that the ball screw 325 rotates with the rotation of the first servo motor 323 and thus the sliding member 34 coupled to the ball nut 365 slides in the first direction X. Meanwhile, known rack or pinion tools may be used instead of the ball screw 325 and the ball nut 365.

The second servo motor 341 is disposed in a direction perpendicular to the first servo motor 323 and is fixed to the sliding member 34.

The frame 36 is slidably coupled to the sliding 34 in a second direction Y along the guide rails 331. The frame 36 includes a through hole 361 through which radiation applied by the device for applying radiation 20 passes. The through hole 361 is disposed to correspond to the first through unit. A plurality of multi-leaf collimators (MLCs) 363 (or shield leafs) is installed in the through hole 361. The MLCs 363 are slidable with respect to each other while each having the same non linear cross sectional structure as shown in FIG. 5. In the present embodiment, the MLCs 363 are separated into two groups at either side of the through hole 361, each group including 25 MLCs. The MLCs 363 may be slidable with respect to the frame 36. The sides of the frame 36 are opened so that portions of one end of the MLCs 363 are pushed or pulled. The MLCs 363 are formed of carbon steel or tungsten alloy that is a material used to shield the radiation applied by the device for applying radiation 20 in order to open and close the radiation if required. The MLCc 363 may be manually operated. The collimator driving unit 40 may further comprise a template 365 for establishing the shape of a radiation passing area determined according to the open shape of the MLCs 363. The template 365 may be formed of an acrylic material. A variety of shapes of the template 365 are previously manufactured so that the shape of the template 365 corresponds to the shape of the patient's treatment part. When the MLCs 363 manipulate the through hole 361 to be opened, if the template 365 is disposed in the through hole 361 and the MLCs 363 slide so as to allow the template 365 to shield the through hole 361, a part of the through hole 361 where the template 365 is disposed is open and the other parts of the through hole 361 are shielded so that the radiation passes in the shape of the patient's treatment part that is to be treated.

The first direction X and the second direction Y are perpendicular to each other. Therefore, the frame 36 is disposed to move two-dimensionally with respect to the body 32 via the sliding member 34. The frame 36 is coupled to the second servo motor 341 in a powered manner. In the present embodiment, like the powered coupling member between the sliding member 34 and the first servo motor 323, the ball screw 326 and the ball nut 367 are used to couple the frame 36 and the second servo motor 341 in a powered manner.

The motor controller 50 is used to generate a signal for controlling the driving of the first servo motor 323 and the second servo motor 341. The motor controller 50 is electrically coupled to the first servo motor 323 and the second servo motor 341 by using an electric wire. The motor controller 50 externally receives position displacement data regarding a motion of the patient's treatment part according to a patient's breathing and generates the signal for controlling the driving of the first servo motor 323 and the second servo motor 341 based on the position displacement data so that the MLCs 363 follow the patient's treatment part and continuously apply the radiation to the patient's treatment part. The position displacement data regarding the patient's treatment part received by the motor controller 50 may be obtained by a device disclosed in the Korean Patent No. 0706758 that is previously filed and granted in the name of the inventors of the present invention. The present invention does not provide the position displacement data regarding the patient's treatment part and thus detailed description of obtaining the position displacement data will not be provided here and reference to the above mentioned patent publication should be made for the detailed description.

Hereinafter, the operation of the radiotherapy apparatus 10 of the present invention will now be described in detail.

The description will be made in the case where a patient lies on the radiotherapy apparatus 10 as shown in FIG. 2. It is assumed that a patient's lung is treated. A shape of radiation applied to the patient's treatment part is manufactured with the template 365. Since the template 365 is formed of an acrylic material, the template 365 can be easily and quickly manufactured. The through hole 361 is opened, the template 365 is inserted into the through hole 361 and the MLCs 363 are pushed toward the center of the through hole 361. The through hole 361 is almost shielded by the MLCs 363 and thus radiation passes through a shaped part of the template 365 as shown in FIG. 3. In this regard, the device 20 for applying radiation applies radiation to the patient's treatment part through the template 365. The template 365 formed of the acrylic material does not greatly influence the progress of radiation applied by the device 20 for applying radiation, which is considered when an applied energy and radiation dose are planned. The position displacement data regarding the patient's treatment part is input in real-time into the motor controller 50 simultaneous with the application of radiation from the device 20 for applying radiation. The position displacement data regarding the patient's treatment part is obtained by using the device disclosed in the Korean Patent No. 0706758 and previously obtained motion information and is input into the motor controller 50. The position displacement data is not input in real-time into the motor controller 50 and may be input into the motor controller 50 in accordance with a predetermined time, e.g. a schedule treatment time. The motor controller 50 generates the signal for controlling the driving of the first servo motor 323 and the second servo motor 341 in order to maintain the radiation application shape formed in the through hole 361 of the frame 36 while the frame 36 continuously follows a motion of the patient's treatment part. As a result, the patient's treatment part is continuously irradiated in the almost same manner as the patient does not breathe. The radiotherapy apparatus 10 of the present embodiment continuously applies radiation to the patient's treatment part while following the patient's treatment part, thereby conducting efficient and quick radiotherapy. Also, the radiotherapy apparatus 10 of the present embodiment does not apply radiation to a patient's non cancerous parts, thereby reducing a side effect of radiotherapy. In the present embodiment, the MLCs 363 are manually manipulated, thereby reducing manufacturing cost of the collimator device 30 for radiotherapy. In the present embodiment, the template 365 is used to determine the shape of the patient's treatment part, thereby precisely setting the patient's treatment part. Although the radiotherapy apparatus 10 comprises the device 20 for applying radiation and the collimator device 30 for radiotherapy, since the collimator device 30 for radiotherapy is easily separated from and attached to the device 20 for applying radiation, the collimator device 30 for radiotherapy can be attached to various devices for applying radiation, thereby remarkably increasing flexibility.

Hereinafter, the specification of a sample product used to verify the effect of the present invention and a test result will now be described.

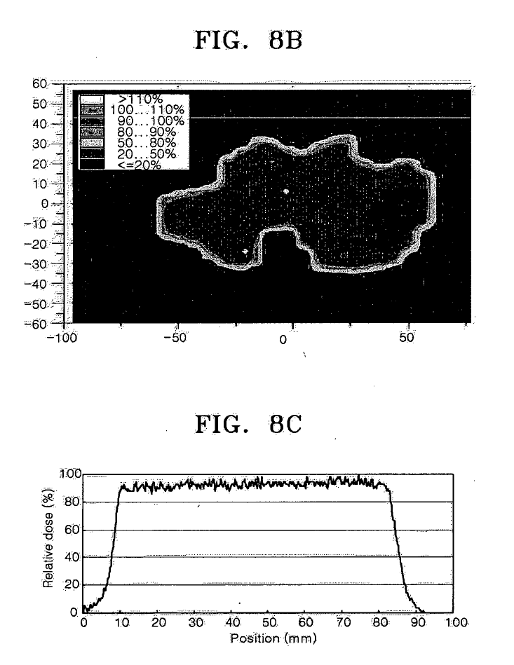

FIG. 6 is a table of specification of the collimator device 30 for radiotherapy. The MLCs 363 of the collimator device 30 for radiotherapy are formed of carbon steel that is an alloy of steel and carbon, includes 50 metal leaves (MLCs) that are separated into two groups each group including 25 leaves. Each of the MLCs 363 may be movable. Each MLC 363 is designed to be 10 cm×0.4 cm×5 cm (length×width×height) and its maximum irradiation surface (or through hole) is designed as 10×10 cm². The side surface of each MLC 363 is designed to be parallel to an applied beam of radiation. The MLCs 363 are manufactured to be adjacent to each other while each having the same non linear cross sectional structure as shown in FIG. 5 in order to prevent leakage of radiation between leafs.

In order to verify usefulness of the collimator device 30 for radiotherapy, radiation was applied to a conventional Lipowitz alloy shield and the collimator device 30 for radiotherapy and images irradiated onto films were compared. A Gafchromic EBT film was used to measure an amount of beam, and was read by using a transparent scanner.

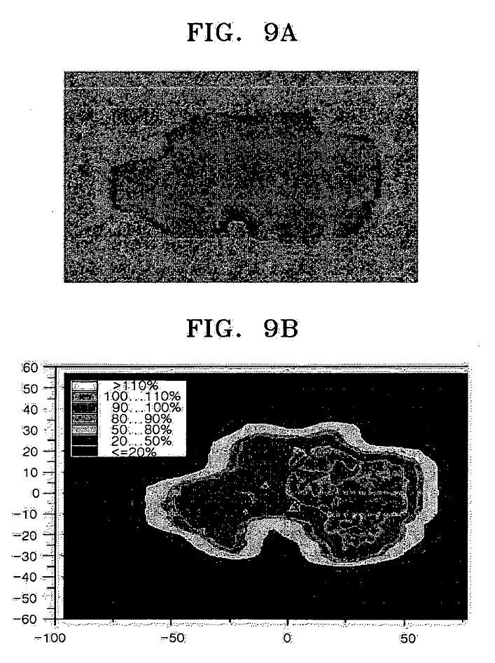

Co-60 was used as a beam source of T780 (AECL, Canada) that is a gamma ray emitting device. A dose rate was 160.76 cGy/min. A distance (SCD) between a radiation source and the collimator device 30 for radiotherapy was 80 cm. A distance (SFD) between the radiation source and the film was 112 cm. The film was fixed by using acryl. FIG. 7 is a table of specific test condition for verifying usefulness of the collimator device 30 for radiotherapy of the present embodiment.

Three tests were conducted. First, when no organ moves, for example, provided that the patient does not exhibit any living body activity, radiation is applied. Second, provided that there is an actual motion of an organ, a film is installed in the device for reproducing a motion of an organ disclosed in the Korean Patent No. 0740339 in the name of the inventors of the present invention based on position displacement data of an organ, and radiation is applied. Third, radiation is applied by using the motion of the organ, the collimator device 30 for radiotherapy, and the device for reproducing the motion of the organ, and a distribution of radiation applied to a film is analyzed.

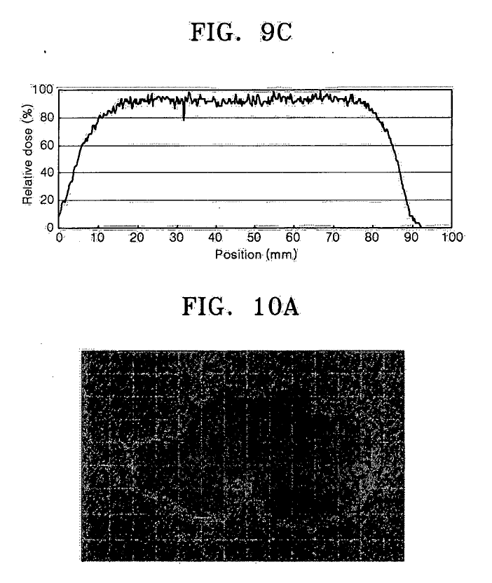

FIGS. 8A through 10B illustrate results of the three tests. Referring to FIGS. 8A through 8C, in order to determine influence on a normal tissue after radiotherapy when no organ moves, a position of a stage in which the film was disposed was fixed, radiation was applied to the film, and an image was obtained. As a result, a clear outline was detected as shown in FIG. 8A.

An isodose curve and a penumbra are detected from a resultant image in order to obtain quantitative data. PTW manufactured by Verisoft is used to analyze the quantitative data. An actual distance regarding the penumbra is measured by obtaining a pixel value of a corresponding point. The test condition is that a pixel is 0.2647857 mm. An average optical density of the image obtained from the first test in a horizontal direction is 157.3 MU. FIG. 8B illustrates the distribution of dose in the film. The range of the penumbra indicating a distance between a maximum dose and 90% and 20% of the isodose curve is 4.8 mm from the left of the isodose curve shown in FIG. 8C and 4.2 mm from the right thereof.

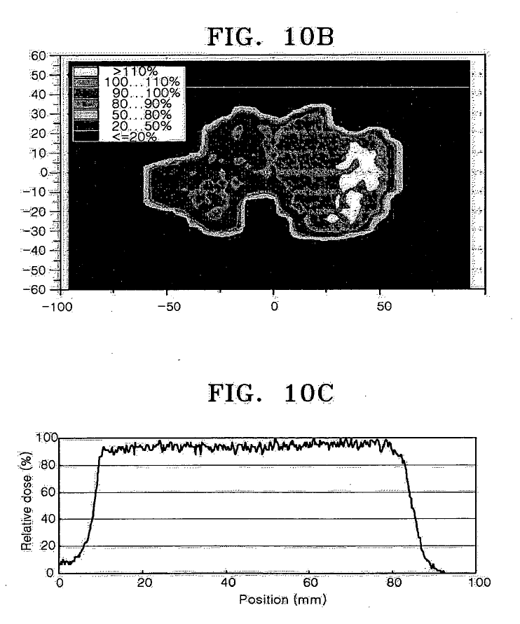

Meanwhile, referring to FIGS. 9A through 9C, an image is obtained by applying radiation to the EBT film after automatically moving the position of the stage in which the film was disposed by a designated range by using the motion of the organ and the signal.

Referring to FIG. 9A, the image has an unclear outline. This means that radiation was not appropriately applied to a range planned for an actual treatment and was thus applied to normal non cancerous parts of an organ. An isodose curve and penumbra are detected from the resultant image in order to obtain more quantitative data.

When a target moves together with a motion signal of the organ, an average optical density of the image in a horizontal direction is 158 MU. FIG. 9B illustrates the distribution of dose in the film. The range of the penumbra is 10.3 mm from the left of an isodose curve shown in FIG. 9C and 13.5 mm from the right thereof. In comparison of the film (i.e., the target) that does not move, errors of 5.5 mm from the left and 9.3 mm from the right occur.

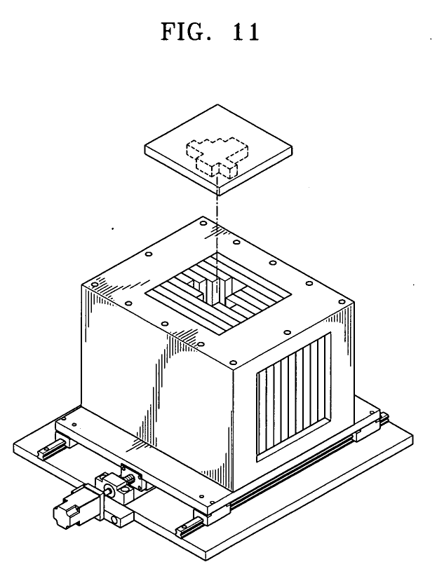

Referring to FIGS. 10A through 10C, an unnecessary exposure of radiation of adjacent tissues is determined by conducting the two tests. In this test, the device for reproducing the motion of the organ disclosed in the Korean Patent No. 0740339 and the collimator device 30 for radiotherapy are used to reduce radiation applied to adjacent tissues. Since radiation is not directly applied to a human being, the test is conduced by using the device for reproducing the motion of the organ as performed in the previous two tests.

FIG. 10A is an image obtained by simultaneously moving the collimator device 30 for radiotherapy and a stage to which a film that is a substitute of motion of an organ is attached and applying radiation, in which a clear outline as shown in FIG. 8A is detected from the image. An average optical density of the image in a horizontal direction is 158 MU. FIG. 10B illustrates the distribution of dose in the film. The range of the penumbra is 6.6 mm from the left of an isodose curve shown in FIG. 10C and 4.2 mm from the right thereof. In comparison of a case where the collimator device 30 for radiotherapy does not simultaneously move, errors of 3.8 mm from the left and 9.3 mm from the right occur. Therefore, if the collimator device 30 for radiotherapy and the motion of the organ are used together during radiotherapy, a treatment error is sure to be reduced.

The body 32 includes the guide rails 321 and the frame 36 is installed to slide along the guide rails 321 in the present embodiment. However, the guide rails 321 may not be formed in the body 32, for example, the guide rails 321 are formed on the frame 36 or the frame 36 is slidable with respect to the body 32 according to another structure.

The MLCs 363 are formed of carbon steel or tungsten alloy in the present embodiment. However, the present invention is not limited thereto and various modifications of the MLCs 363 for shielding radiation may be used.

The MLCs 363 are manually manipulated in the present embodiment. However, the MLCs 363 may be automatically manipulated by using a motor or a gear, although this does increase manufacturing costs.

The MLCs 363 are slidable by the correlations between the adjacent collimators and the unevenness structure in the present embodiment. However, although the MLCs 363 do not have the unevenness structure, for example, the MLCs 363 may surface contact each other so that the MLCs 363 are slidable with respect to each other.

The collimator driving unit 40 further comprises the template 365 for establishing the shape of the radiation passing area and the template 365 is formed of an acrylic material in the present embodiment. However, the template 365 may be formed of various materials as long as it is a material such as wood that does not shield radiation, and the template 365 may not be included in the collimator driving unit 40.

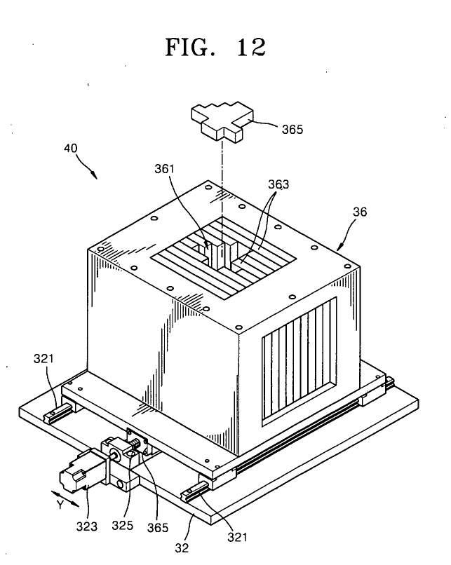

The sliding member 34 is movably coupled to the body 32 in the first direction. The frame 36 is movably coupled to the sliding member 34 in the second direction perpendicular to the first direction with respect to the body 32. The first servo motor 323 is installed in the body 32 and is coupled to the sliding member 34 in a powered manner in order to move the sliding member 34 in the first direction with respect to the body 32. The second servo motor 341 is installed in the sliding member 34 and is coupled to the frame 36 in a powered manner in order to move the frame 36 in the second direction with respect to the sliding member. However, referring to FIG. 11, a frame is movably coupled to move in a direction with respect to a body, and a servo motor is installed in the frame and is coupled to the frame in a powered manner in order to move the frame in the direction with respect to the body.

The collimator device for radiotherapy and radiotherapy apparatus according to the present invention follow the patient's treatment part and continuously and precisely apply radiation to the patient's treatment part, thereby efficiently and quickly treating a patient.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A collimator device for radiotherapy comprising:
a body including a first through unit and disposed on a path of high energy radiation which in use is irradiated toward a patient's treatment part;
a frame including a through hole corresponding to the first through unit and slidably installed in the body;
a plurality of multi-leaf collimators (MLCs) slidably installed in the through hole and including radiation shields;
a servo motor coupled to the body and the frame in a power manner so as to slidingly move the frame with respect to the body; and
a motor controller externally receiving position displacement data regarding a motion of the patient's treatment part due to a patient's breathing and generating a signal for controlling the driving of the servo motor so that the MLCs follow the patient's treatment part and continuously apply radiation to the patient's treatment part based on the position displacement data.

2. The collimator device for radiotherapy of claim 1, wherein the body comprises two guide rails,
wherein the frame is slidable along the two guide rails.

3. The collimator device for radiotherapy of claim 1, wherein the radiation shields are formed of carbon steel or tungsten alloy.

4. The collimator device for radiotherapy of claim 1, wherein the MLCs are manually manipulated.

5. The collimator device for radiotherapy of claim 1, wherein the MLCs are slidable by the correlation coupling of adjacent MLCs and each having a non linear cross sectional structure.

6. The collimator device for radiotherapy of claim 1, further comprising: a template establishing the shape of a radiation passing area of the MLCs and formed of an acrylic material.

7. The collimator device for radiotherapy of claim 1, wherein the frame is movably coupled to move in a direction with respect to the body,
wherein the servo motor is installed in the frame and is coupled to the frame in a powered manner so as to move the frame in the direction with respect to the body.

8. The collimator device for radiotherapy of claim 1, wherein the body comprises a sliding member movably coupled to the body in a first direction,
wherein the frame is movably coupled to the sliding member in a second direction perpendicular to the first direction with respect to the sliding member,
wherein the servo motor comprises a first servo motor installed in the body and coupled to the sliding member in a powered manner so as to move the sliding member in the first direction with respect to the body, and a second servo motor installed in the sliding member and coupled to the frame in a powered manner so as to move the frame in the second direction with respect to the sliding member.

9. A radiotherapy apparatus comprises:
a device for applying radiation;
a body including a first through unit, disposed on a path of high energy radiation which in use is irradiated toward a patient's treatment part, and coupled to the device for applying radiation;
a frame including a through hole corresponding to the first through unit and slidably installed in the body;
a plurality of MLCs slidably installed in the through hole and including radiation shields;
a servo motor coupled to the body and the frame in a powered manner so as to slidingly move the frame with respect to the body; and
a motor controller externally receiving position displacement data regarding a motion of the patient's treatment part due to a patient's breathing and generating a signal for controlling the driving of the servo motor so that the MLCs follow the patient's treatment part and continuously apply radiation to the patient's treatment part based on the position displacement data.
